(19) United States
(12) Patent Application Publication
(10) Pub. No.: US 2012/0209135 A1
(43) Pub. Date: Aug. 16, 2012

(54) Implantable Device, System and Method for Measuring Physiologic Parameters

(76) Inventor: Mark Zdeblick, Portola Valley, CA (US)

(21) Appl. No.: 13/318,116

(22) PCT Filed: Apr. 29, 2010
(86) PCT No.: PCT/US10/33040

§ 371 Date: May 4, 2012

Related U.S. Application Data
(60) Provisional application No. 61/174,480, filed on Apr. 30, 2009.

(51) Int. Cl. A61B 5/053

(52) U.S. Cl. 600/547

(57) ABSTRACT

An apparatus having at least one implantable lead, e.g., two leads, three leads, etc., is provided. In one example, the apparatus includes at least two satellites electrically coupled to a common implantable pulse generator by at least two conductors extending from the common implantable pulse generator to each of the at least two satellites. Each satellite, for example, is associated with a respective tissue site such as a cardiac wall.

This patent claims the benefit of prior U.S. Provisional Patent Application No. 61/174,481, filed Apr. 30, 2009 and titled “Implantable Device, System, and Method for Measuring Physiologic Parameters”, which patent application is incorporated herein by reference for all purposes.

Implantable medical devices (IMDs) are devices designed to be implanted into a patient. These devices may be associated with different body parts, e.g., bladder, stomach, heart, etc. Examples of implantable devices include cardiac function management (CFM) devices, drug delivery systems, neurological simulators, bone growth simulators, etc. CFMs include implantable pacemakers, implantable defibrillators (ICDs), and devices that include a combination of pacing and defibrillation to facilitate different treatment methods, e.g., cardiac resynchronization therapy. These devices are typically used to treat patients, e.g., via electrical therapy, and to aid a physician or caregiver in patient diagnosis through constant monitoring of a patient's condition.

Conventional methods employed by the devices, however, may render unreliable results. For example, measurements taken by the devices may prove too ambiguous to accurately represent a physiologic parameter.

It is desirable, therefore, to produce improved devices, systems and methods for monitoring various physiologic parameters from which to derive various clinical information.

Therefore, it would be an important advancement in medical therapies to derive such clinical information by making use of technologically-advanced devices that use modern techniques. Such devices may be used in various technology areas including managed health care.

### BRIEF DESCRIPTION OF THE DRAWINGS

The invention will be described with respect to the following:

FIG. 1 shows an implantable device and leads implanted in a human heart.

FIG. 2 shows a lead having satellites, the lead of a type employed in FIG. 1 and an expanded view of a satellite.

FIG. 3 represents various values of resistance associated with various areas of the human heart.

FIG. 4 depicts portions of two integrated circuits associated with satellites of a lead.

FIG. 5 depicts conductors connecting two integrated circuits of the type shown in FIG. 4.

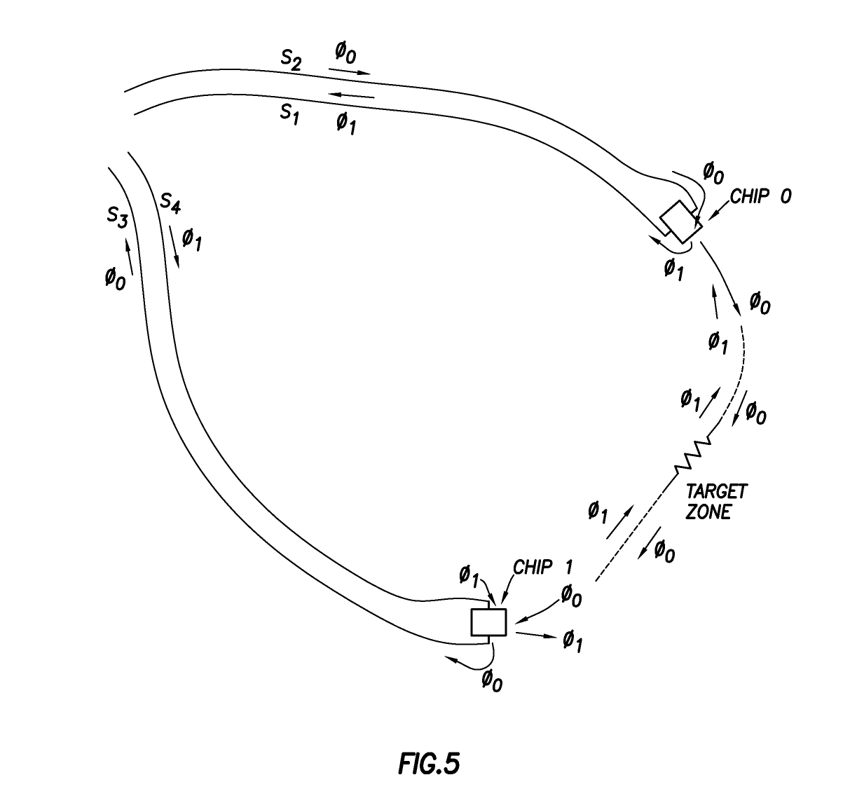

Where possible, like elements among the figures have been designated with like reference numerals, for convenience of notation.

### DETAILED DESCRIPTION

Various aspects provide an apparatus having at least one implantable lead, e.g., two leads, three leads, etc. In one example, the apparatus includes at least two satellites electrically coupled to a common implantable pulse generator by at least two conductors extending from the common implantable pulse generator to each of the at least two satellites. Each satellite, for example, is associated with a respective tissue site such as a cardiac wall.

Each of the satellites includes, for example, an integrated circuit coupled to the two conductors and having a differential amplifier, a first electrode communicatively coupled to an output of the integrated circuit, the first electrode selectively associated with a first portion of a tissue site, and a second electrode adjacent to the first electrode communicatively coupled to an input of the integrated circuit, the second electrode selectively associated with a second portion of a tissue site.

The respective differential amplifier generates predetermined voltages between the second electrode of the first satellite and the second electrode of the second satellite enabling measurement of electrical impedance of tissue substantially between the first tissue site and the second tissue site.

In various aspects, the voltage on the second electrode of a particular satellite provides feedback to the differential amplifier which produces a drive voltage on the first electrode of the particular satellite.

In various aspects, the second input of the differential amplifier in a particular satellite is a reference parameter generated by the corresponding integrated circuit. The reference parameter may comprise, for example, a reference voltage or a reference current.

Further, in some aspects, the reference parameter in the first satellite may be a parameter substantially equivalent to, and of opposite polarity to, a reference parameter of the second satellite.

In other aspects, the reference parameter in the first satellite may be a time-varying reference parameter and the reference parameter of the second satellite may be a substantially non-time-varying reference voltage.

For example, the time-varying reference parameter may be substantially a sine wave, substantially a triangle wave, or substantially a square wave.

In various aspects, the reference parameter is a voltage that is a multiple of a diode drop, e.g., one diode, two diodes, etc.

In various aspects, the at least one electrode comprises a segmented electrode, e.g., a two-segmented electrode, a four-segmented electrode, etc.

Various employments and applications apply. To illustrate, apparatus may be associated with various body tissues, e.g., organs such as a lung, a heart, a bladder, and a stomach.

In various aspects, a system includes an implantable pulse generator (IPG) and at least one implantable lead. The implantable leads includes, for example, two or more satellites electrically coupled to the IPG by at least two conductors extending from the IPG to each of the at least two satellites.

Each of the satellites may be associated with a respective tissue site, e.g., a first satellite associated with a first tissue site and a second satellite associated with a second tissue site.

Each of the satellites may include, for example, an integrated circuit coupled to the two conductors, a differential amplifier, a first electrode communicatively coupled to an output of the integrated circuit, and a second electrode adjacent to the first electrode and communicatively coupled to an input of the integrated circuit.

The first electrode may be selectively associated with a first portion of a tissue site. The second electrode may be selectively associated with a second portion of a tissue site.

The respective differential amplifiers may generate predetermined voltages between the second electrode of the first satellite and the second electrode of the second satellite which may enable measurement of electrical impedance of tissue substantially between the first tissue site and the second tissue site.

In some aspects, the second input of the differential amplifier in a particular satellite may be a reference parameter generated by the corresponding integrated circuit.

In some aspects, the reference parameter may be a reference voltage or a reference current.

In various aspects, the common implantable pulse generator includes a current sense circuit that measures the time-varying current between the two conductors.

In various aspects, the common implantable pulse generator includes a voltage sense circuit that measures the time-varying voltage between the two conductors.

In some aspects, the pulse generator includes an analog-to-digital converter communicably associated with the integrated circuit and an amplifier communicably associated with the analog-to-digital converter.

In some aspects, an apparatus includes one or more implantable leads. The implantable lead includes one or more satellites electrically coupled to a power source by two or more conductors extending from the power source to each of the satellites.

Each satellite may be associated with a respective tissue site, e.g., a first satellite associated with a first tissue site, a second satellite associated with a second tissue site, etc.

In various aspects, each satellite includes an integrated circuit coupled to the two conductors and having a variable current regulator and a voltage sampling circuit. Each satellite further includes a first electrode communicatively coupled to an output of the variable current regulator, the first electrode selectively associated with a first portion of a tissue site and a second electrode adjacent to the first electrode communicatively coupled to an input of the voltage sampling circuit, the second electrode selectively associated with a second portion of a tissue site.

The voltage sampling circuits may be capable of transmitting data representing the voltages, the voltages from the first satellite and the second satellite represent the electrical impedance of tissue substantially between the first tissue site and the second tissue site. To illustrate, the data may be used to calculate a voltage difference, where the voltage difference is used as an input to calculate impedance.

In some aspects, the variable current in the first satellite may be substantially equivalent to, but of opposite polarity to, a variable current of the second satellite.

In some aspects, the power source may provide a signal that synchronizes the current sources on each of the two satellites.

In some aspects, the variable current in the first satellite is regulated and the variable current in the second satellite is not regulated.

In some aspects, the first current source may vary independently of the second satellite.

In various aspects, a method is provided for use with an apparatus comprising two or more satellites electrically coupled to a common implantable pulse generator by two or more conductors extending from the common implantable pulse generator to each of the satellites.

Each satellite may be associated with a respective tissue site. The satellites may include, for example, a first satellite associated with a first tissue site and a second satellite associated with a second tissue site.

Each satellite may further include an integrated circuit coupled to the two conductors, a differential amplifier, a first electrode communicatively coupled to an output of the integrated circuit, and a second electrode adjacent to the first electrode and communicatively coupled to an input of the integrated circuit.

The first electrode may be selectively associated with a first portion of a tissue site.

The second electrode may be selectively associated with a second portion of a tissue site.

In various aspects, the method includes a step of generating predetermined voltages between the second electrode of the first satellite and the second electrode of the second satellite enabling measurement of electrical impedance of tissue substantially between the first tissue site and the second tissue site.

In various aspects, a system includes an implantable pulse generator. The implantable pulse generator includes, for example, a variable current source capable of providing a time-varying current through each of two conductors and one or more implantable leads.

The implantable lead(s) may include, for example, two or more satellites electrically coupled to the pulse generator by at least two conductors extending from the implantable pulse generator to each of the at least two satellites.

Each satellite may be associated with a respective tissue site.

To illustrate, a first satellite of the at least two satellites may be associated with a first tissue site. The first satellite may include an integrated circuit coupled to the two conductors and having a voltage sampling circuit, a first electrode communicatively connected to a first conductor, and a second electrode adjacent to the first electrode and communicatively coupled to an input of the voltage sampling circuit. The first electrode may be selectively associated with a first portion of the first tissue site. The second electrode may be selectively associated with a second portion of the first tissue site. A second satellite of the at least two satellites may be associated with a second tissue site. The second satellite may include, for example, an integrated circuit coupled to the two conductors and having a voltage sampling circuit, a third electrode communicatively connected to a second conductor, the third electrode selectively associated with a first portion of the second tissue site; and a fourth electrode adjacent to the third electrode and communicatively coupled to an input of the voltage sampling circuit of the second satellite, the fourth electrode selectively associated with a second portion of the second tissue site. The voltage sampling circuits may be capable of transmitting data representing the voltages. The data and/or voltages from the first satellite and the second satellite may represent the electrical impedance of tissue substantially between the first tissue site and the second tissue site.

In some aspects, the integrated circuits may scavenge power from the time-varying current provided by the implantable pulse generator.

In some aspects, the integrated circuits may count the number of cycles delivered by the implantable pulse generator.

In some aspects, each integrated circuit may transmit a number representing a particular electrode voltage associated with the integrated circuit after a particular number of cycles have been transmitted.

With reference now to the Figures:

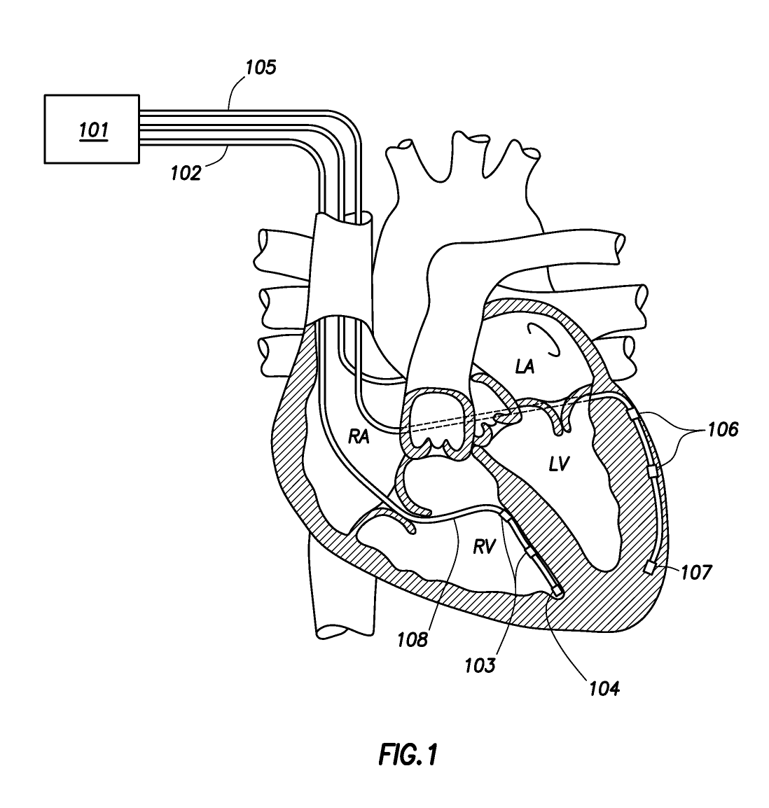

An implantable pulse generator according to an aspect of the invention is depicted in FIG. 1. FIG. 1 illustrates locations of a number of pacing satellites incorporated in multi-electrode pacing leads, in accordance with an aspect of the present invention. A pacing and signal detection system 101 provides extra-cardiac communication and control elements for the overall system. In some aspects, pacing and signal detection system 101 may be, for example, a pacing can of a pacemaker residing in an external or extra-corporeal location.

Right ventricular lead 102 emerges from pacing and signal detection system 101 and travels from a subcutaneous location from pacing and signal detection system 101 into the patient's body (e.g., preferably, a subclavian venous access), and through the superior vena cava into the right atrium. From the right atrium, right ventricle lead 102 is threaded through the tricuspid valve to a location along the walls of the right ventricle. The distal portion of right ventricular lead 102 is preferably located along the intra-ventricular septum, terminating with a fixation in the right ventricular apex. Right ventricular lead 102 includes satellites positioned at locations 103 and 104. The number of satellites in ventricular lead 102 is not limited, and may be more or less than the number of satellites shown in FIG. 1.

Similarly, left ventricular lead 105 emerges from the pacing and signal detection system 101, following substantially the same route as right ventricular lead 102 (e.g., through the subclavian venous access and the superior vena cava into the right atrium). In the right atrium, left ventricular lead 105 is threaded through the coronary sinus around the posterior wall of the heart in a cardiac vein draining into the coronary sinus. Left ventricular lead 105 is provided laterally along the walls of the left ventricle, which is likely to be an advantageous position for bi-ventricular pacing. FIG. 1 shows satellites positioned at locations 106 and 107 along left ventricular lead 105. Right ventricular lead 102 may optionally be provided with pressure sensor 108 in the right ventricle. A signal multiplexing arrangement allows a lead to include such active devices (e.g., pressure sensor 108) for pacing and signal collection purposes (e.g., right ventricular lead 102). Pacing and signal detection system 101 communicates with each of the satellites at locations 103, 104, 106 and 107. The electrodes controlled by the satellites may also be used to detect cardiac depolarization signals. Additionally, other types of sensors, such as an accelerometer, strain gauge, angle gauge, temperature sensor, can be included in any of the leads.

In the above system, the device components can be connected by a multiplex system (e.g., as described in United States Patent publication no. US 20040254483 entitled “Methods and systems for measuring cardiac parameters”; US patent publication number US 20040220637 entitled “Method and apparatus for enhancing cardiac pacing”; US patent publication number US 20040215049 entitled “Method and system for remote hemodynamic monitoring”; US patent publication number US 20040193021 entitled “Method and system for monitoring and treating hemodynamic parameters; the disclosures of which are herein incorporated by reference), to the proximal end of electrode lead 105. The proximal end of electrode lead 105 connects to a pacemaker 101, e.g., via an IS-1 connector.

Devices of invention may include a multiplexed multi-electrode component. Multiplexed multi-electrode components include two or more electrodes which are electrically coupled, either directly or through an intermediate connector, to a common conductor or set of common conductors, such that the two or more electrodes share one or more conductors. The term “conductor” refers to a variety of configurations of electrically conductive elements, including wires, cables, etc. A variety of different structures may be implemented to provide the multiplex configuration. Multiplex configurations of interest include, but are not limited to, those described in: PCT application no. PCT/US2003/039524 published as WO 2004/052182; PCT application no. PCT/US2005/031559 published as WO 2006/029090; PCT application no. PCT/US2005/046811 published as WO 2006/069322; PCT application no. PCT/US2005/046815 published as WO 2006/069323; and PCT application no. PCT US2006/048944 published as WO 2007/075974; the disclosures of which are herein incorporated by reference.

During certain aspects of use, the electrode lead 105 is placed in the heart using standard cardiac lead placement devices which include introducers, guide catheters, guidewires, and/or stylets. Briefly, an introducer is placed into the clavicle vein. A guide catheter is placed through the introducer and used to locate the coronary sinus in the right atrium. A guidewire is then used to locate a left ventricle cardiac vein. The electrode lead 105 is slid over the guidewire into the left ventricle cardiac vein and tested until an optimal location for CRT is found. Once implanted a multi-electrode lead 105 still allows for continuous readjustments of the optimal electrode location. The electrode lead 102 is placed in the right ventricle of the heart. In this view, the electrode lead 102 is provided with one or multiple electrodes 103, 104.

Electrode lead 102 is placed in the heart in a procedure similar to the typical placement procedures for cardiac right ventricle leads. Electrode lead 102 is placed in the heart using the standard cardiac lead devices which include introducers, guide catheters, guidewires, and/or stylets. Electrode lead 102 is inserted into the clavicle vein, through the superior vena cava, through the right atrium and down into the right ventricle. Electrode lead 102 is positioned under fluoroscopy into the location the clinician has determined is clinically optimal and logistically practical for fixating the electrode lead 102.

Summarizing aspects of the above description, in using the implantable pulse generators of the invention, such methods include implanting an implantable pulse generator e.g., as described above, into a subject; and the implanted pulse generator, e.g., to pace the heart of the subject, to perform cardiac resynchronization therapy in the subject, etc. The description of the present invention is provided herein in certain instances with reference to a subject or patient. As used herein, the terms “subject” and “patient” refer to a living entity such as an animal. In certain aspects, the animals are “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g. rabbits) and primates (e.g., humans, chimpanzees, and monkeys). In certain aspects, the subjects, e.g., patients, are humans.

During operation, use of the implantable pulse generator may include activating at least one of the electrodes of the pulse generator to deliver electrical energy to the subject, where the activation may be selective, such as where the method includes first determining which of the electrodes of the pulse generator to activate and then activating the electrode. Methods of using an IPG, e.g., for pacing and CRT, are disclosed in U.S. Pat. No. 7,214,189 entitled “Methods and apparatus for tissue activation and monitoring”; US publication number US 2008-0255647 entitled “Implantable addressable segmented electrodes”; PCT publication number WO 2006/069323 entitled “Implantable hermetically sealed structures”; international patent publication number WO 2007/075974 entitled “Implantable integrated circuit”; and US patent publication US 2008-0077186 entitled “High phrenic, low capture threshold pacing devices and methods”; the disclosures of the various methods of operation of these applications being herein incorporated by reference and applicable for use with the present devices.

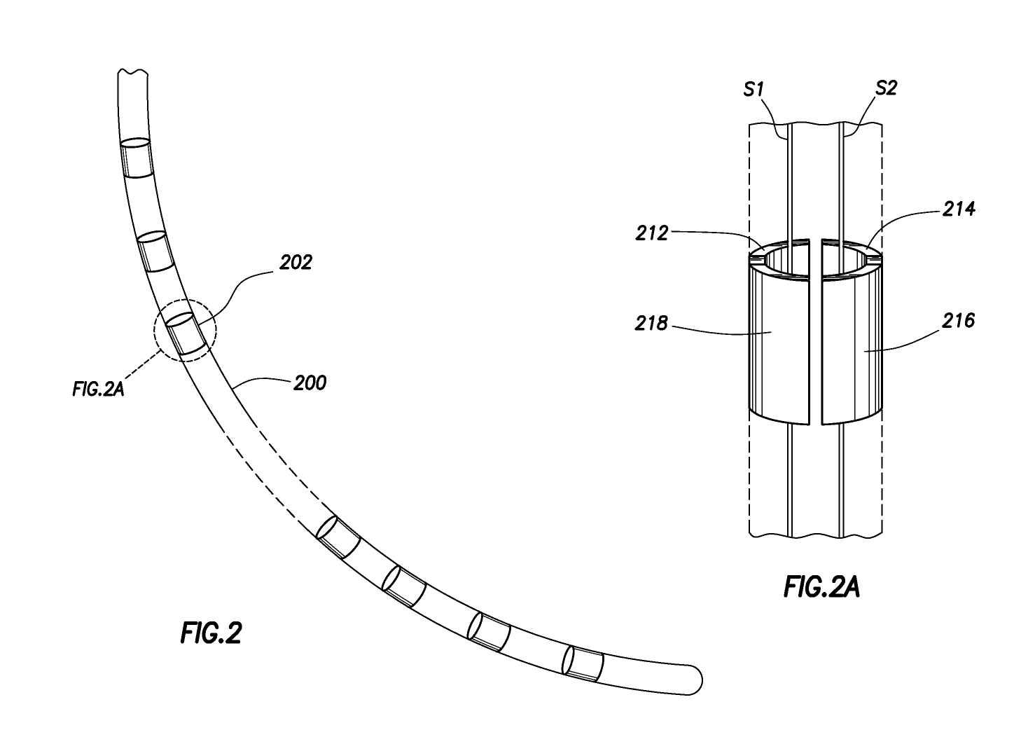

FIG. 2 is an external view of a number of exemplary pacing satellites, in accordance with a multiplex lead aspect of the present invention. According to one aspect, a pacing lead 200 (e.g., right ventricular lead 102 or left ventricular lead 105 of FIG. 1) accommodates two bus wires S1 and S2, which are coupled to a number (e.g., eight) of satellites, such as satellite 202. S2, in an exemplary aspect, is an anode conductor, and S1 is a cathode conductor. FIG. 2 also shows satellite 202 with an enlarged view. Satellite 202 includes electrodes 212, 214, 216, and 218, located in the four quadrants of the cylindrical outer walls of satellite 202 and supported by a support structure of the invention. Each satellite also contains a control chip inside the structure which communicates with a pacing and signal-detection system to receive configuration signals that determine which of the four electrodes is to be coupled to bus wires S1 or S2.

The configuration signals, the subsequent pacing pulse signals, and the analog signals collected by the electrodes can all be communicated through bus wires S1 and S2, in either direction. Although shown in a symmetrical arrangement, electrodes 212, 214, 216 and 218 may be offset along lead 200 to minimize capacitive coupling among these electrodes. The quadrant arrangement of electrodes allows administering pacing current via electrodes oriented at a preferred direction, for example, away from nerves, or facing an electrode configured to sink the pacing current. Such precise pacing allows low-power pacing and minimal tissue damage caused by the pacing signal.

The leads may further include a variety of different effector elements, which elements may employ the satellites or structures distinct from the satellites. The effectors may be intended for collecting data, such as but not limited to pressure data, volume data, dimension data, temperature data, oxygen or carbon dioxide concentration data, hematocrit data, electrical conductivity data, electrical potential data, pH data, chemical data, blood flow rate data, thermal conductivity data, optical property data, cross-sectional area data, viscosity data, radiation data and the like. As such, the effectors may be sensors, e.g., temperature sensors, accelerometers, ultrasound transmitters or receivers, voltage sensors, potential sensors, current sensors, etc. Alternatively, the effectors may be intended for actuation or intervention, such as providing an electrical current or voltage, setting an electrical potential, heating a substance or area, inducing a pressure change, releasing or capturing a material or substance, emitting light, emitting sonic or ultrasound energy, emitting radiation and the like.

Effectors of interest include, but are not limited to, those effectors described in the following applications by at least some of the inventors of the present application: U.S. Patent publication number US 20040193021 entitled “Method And System For Monitoring And Treating Hemodynamic Parameters”; U.S. Patent publication number US 20060058588 entitled “Methods And Apparatus For Tissue Activation And Monitoring”; International patent publication number WO 2006/069323; U.S. Patent publication no. US 2006-0161211 entitled “Implantable Accelerometer-Based Cardiac Wall Position Detector”; U.S. Pat. No. 7,200,439 entitled “Method and Apparatus for Enhancing Cardiac Pacing”; U.S. Pat. No. 7,204,798 entitled “Methods and Systems for Measuring Cardiac Parameters”; U.S. Pat. No. 7,267,649 entitled “Method and System for Remote Hemodynamic Monitoring”; U.S. patent publication no. US 2006-0217793 entitled “Fiberoptic Tissue Motion Sensor”; U.S. Pat. No. 7,028,550 entitled “Implantable Pressure Sensors”; U.S. Patent publication No. US 2006-0116581 entitled “Implantable Doppler Tomography System”; and US patent publication US 2008-0255629 entitled “Cardiac Motion Characterization by Strain Measurement”. These applications are incorporated in their entirety by reference herein.

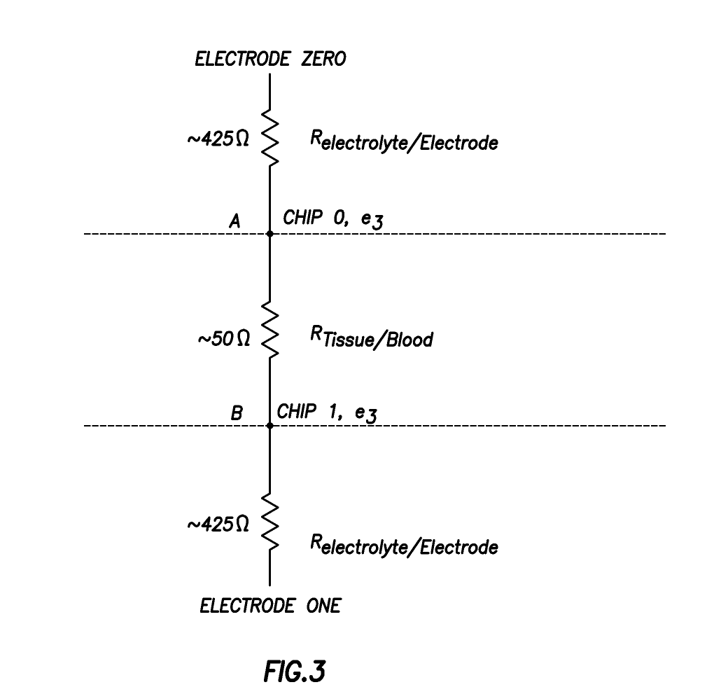

A typical satellite 202 (shown in FIG. 2) is shown in more detail in FIG. 3. Conductors S1, S2 are shown, connecting with an IC (integrated circuit) 301, which is a six-terminal device. The other four terminals are connection points to electrodes 212, 214, 216, and 218 (also visible in FIG. 2).

Additional details regarding individually addressable satellite electrode structures can be found in PCT application serial no. PCT/US2005/031559 published as WO 2006/029090; PCT application serial no. PCT/US2005/046815 published as WO 2006/069323; PCT application serial no. PCT/US2005/046811 published as WO 2006/069322; and U.S. application Ser. No. 11/939,524 published as US 2008-0114230 A1; the disclosures of which are herein incorporated by reference.

FIG. 3 represents various values of resistance associated with various areas of the human heart. To illustrate, consider the human heart having implanted leads (as shown in FIG. 1). Two electrodes of the lead(s), e.g., two electrodes of two satellites of a single lead or one electrode each of one satellite each of two respective leads, are associated with respective tissue sites. The total impedance between these two electrodes is on the order of 1000 Ohms. However, the largest impedance measurement is found between the electrode and the tissue itself and occurs over a very small distance, e.g., between electrode zero and point A as well as between electrode one and point B. This impedance may be on the order of 10 Angstroms or so. This impedance occurs right at the electrode/tissue interface or at the electrode/blood interface and varies in the cardiac cycle by varying the surrounding properties. For example, varying pressure, blood flow rate, and/or other properties affects impedance. This is illustrated by the resistance between electrode zero and point A and between electrode one and point B.

The impedance associated with the remaining zone, e.g., between point A and point B “the target zone”, is on the order of 50 ohms, represented by R Tissue/blood. The one that is actually being measures accurately is only the variation in this 50 ohms, the variation being of the order of plus or minus 10%. Thus, the variation of + or −5 out of the total 1000 is a much smaller percentage than 5 out of 50. It can be seen that for an accurate measurement, it is preferred to measure the variation in 50 Ohms rather than the variation in the 1000.

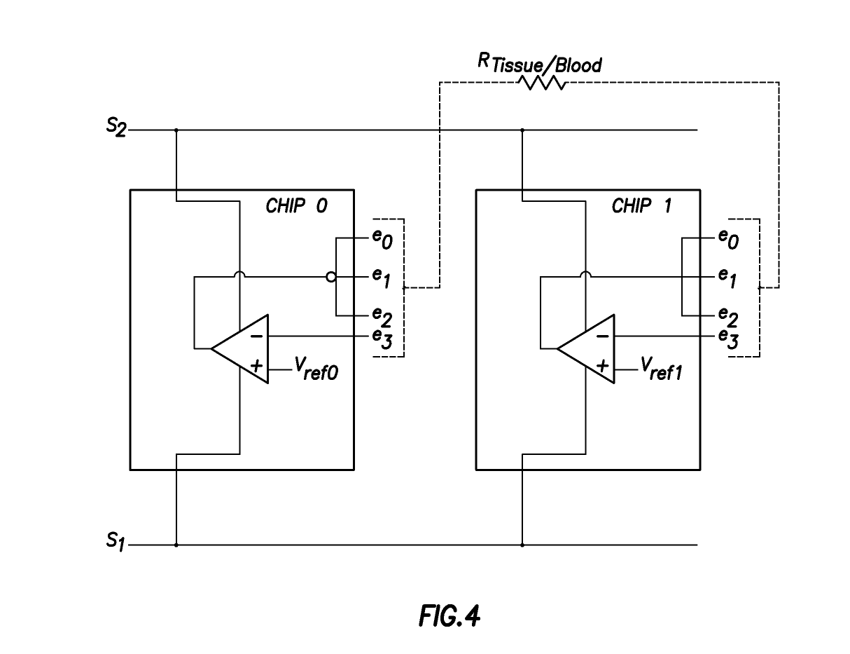

FIG. 4 depicts portions of two integrated circuits associated with satellites of a lead. S1 and S2 are two wires going out of the lead integrated circuit (IC). Measurements may be anywhere between the two different electrodes points, and for the sake of clarity, it is assumed that both wires are on the same two-wire bus. One skilled in the art can realize that because that other components and configurations are possible. The lead ICs/leads are illustrated having an arbitrary connection with S2 and S1.

It is further assumed that on each of ICs, i.e., Chip 0 and Chip 1, the impedance between electrodes are effectively measured. On either IC, current is driven out of the three of the electrodes, e0, e1, and e2, with their gain together. This can be arbitrarily either e0, e1, e2 or some combination thereof. In one aspect, the electrode interface is minimized to tie it to the three electrodes. These electrodes are electronically switched, and shown in FIG. 4 as connected electrically. The fourth electrode, e3 is as left as the input to a differential amplifier, e.g., an operational amplifier (OP-AMP). The op-amp is said to drive in a feedback loop where a reference voltage is applied to the op-amp. There are two available reference voltages. On Chip 0, the input is referenced as VRef0. The associated op-amp drives a current to the three electrodes e0, e1, and e2 in such a way that the input to the op-amp is the same as reference zero. One skilled in the art would realize that one may include some capacitors, resistors and some other well-known circuit elements to implement this technology.

Further, the op-amp may be supplied either directly to S1, S2 or through some sequence of power management systems that provide a stable amount of power to the op-amp. In one aspect, the amount of current that runs the op-amp is of the order of 10 micro-amps. Thus, even when it is not driving current to the electrodes, the op-amp is drawing a current of about 10 micro-amps. The input to the op-amp is e3.

A similar situation may be set up on Chip 1, similar but slightly different. Once again, e0, e1 and e2 are tied together and e3 is used as the input to an op-amp. In this case, the voltage reference is a different reference, e.g., VRef1. The output to this op-amp similarly drives through the electrodes and again, a power to the op-amp is derived from the voltage across S1 and S2. So, for example, one might put 5 volts across S1 and S2. A 10 micro amps current is drawn by each of these two op-amps that are here, and all the other chips are turned off, and are out of the circuit. This, in one aspect both op-amps are driven to VRef0. Since both are at VRef0, there is no current is flowing between the electrodes on Chip 0 and the electrodes on Chip 1 because they are all at the same potential of VRef0.

Therefore, the current flowing in that case between S2 and S1 is approximately 20 micro-amps and the IPG which is driving these voltages is able to measure current of 20 micro-amps which indicates that no current is flowing between the electrodes of Chip 0 and the electrodes of Chip 1. Subsequently, the reference on chip 1 is changed to VRef1, which is a diode drop higher that than reference zero, which is 0.7 v or so. So, VRef 1=VRef 0+0.7 v.

In various aspects, it may be desirable to keep this voltage drop low to reduce the power consumption. It is clear to one skilled in the art that, the bigger the voltage, the more the current will change and the easier and more accurately the impedance may be measured between these two chips. There may be some flexibility as to the actual values of VRef and VRef1, but for the sake of making the circuit simple and easy two choices include having VRef0 one diode drop greater than S1 and VRef1 two diode drops greater than S1. Thus, there is a difference of a well-known, established voltage of about 0.7 v and that voltage would be a function of band gap of the chips, which number is uniform across the silicon wafer and would be stable over time. There is now a 0.7 v drop between Chip 0 and Chip 1 and that is achieved through the sampling of voltage of e3 that is exposed to tissue associated with the target zone, e.g., muscle tissue or blood tissue, and away from the electrode/tissue interface.

Electrode e3 on each of these ICs (sometimes referred to as “chips”) is measuring node A or node B. For example, node A might be Chip 0, e3 and node B might be chip 1: e3 is being sampled here. Also, e3 may be sampling the potentials away from electrodes e0, e1, e2, and away from their associated electrolyte-electrode interface. Thus, current would actually flow between this region, e.g., near Chip 0, then through the path represented by the dotted lines, going to blood and the resistor, and through the resistor which is the resistance of tissue and blood, and this region which is between the electrode and chip 1, i.e., the target zone. VRef1 is greater than VRef0 and thus the current flow is from S2 to Chip 0 of op-amp, out to electrode e0, e1 and e2, through chip tissue to the dotted line resistance and electrodes e0, e1, e2 on Chip 0 and then flows back into the op-amp and out into S1.

Thus, for example, if the resistance of the target zone is 50 ohms, the amount of additional current is the difference of VRef 1 minus VRef0, divided by the impedance, i.e., the tissue-blood impedance. In this example, the current will be 0.7 v divided by 50 ohms which will be equal to 13 milliamps. It is noted that this results in a very large number as compared to 10 micro amps. Such measurements may be carried out for relatively short period of time and at different frequencies. In one aspect, there is an A/C voltage and assume an A/C current. The easiest way of making it an A/C number is by making square waves, sine waves, triangle waves, etc. To illustrate, if the resistance changes from 50 ohms to 55 ohms, there is a new current i2 would equal to 0.7 v divided by 55 which will be 12.7 milli amp. This ratio, i.e., 5/50, is a ratio easily given to accurate measurement (as compared with 5/1000). Other ways are possible.

To illustrate some of the foregoing concepts, consider varying frequencies. There is a sinusoid of a given frequency and a difference in reference voltage. This would be at two different frequencies, one of them would be in the range 10 KHz or 15 KHz, the second one at a different time but very soon thereafter, e.g., 10 micro seconds later, send a second 100 KHz signal.

In one aspect, a sine wave is used. In this case, one measures the peak amplitudes with op-amp that would be tracking these numbers and the current also will be a sinusoidal current with a peak of approximately 14 milliamps depending on the actual measurements. If one does this for a square wave though, one might use a square wave. In this case, each op-amp is switched at exactly the same time from VRef 0 to VRef 1 and vice versa. In one phase, chip 0 would be at VRef0 and chip 1 would be at Vref 1. In the next phase, which is 10 microsec later, 100 Khz is passed and at a higher frequency chip 0 is switched to be VRef1 and chip 1 to be VRef0. In case of a square reference, the op-amp tracks similar references. The actual impedance is switching the a/c but the current at S1 and S2 is actually the DC component of that. However, there would not be the a/c component because S1 and S2 are summing up total current, going through both the chips. If this is done by two separate lines, then one would be seeing current in one branch and in the other branch, the a/c signal of S1, S2 and the a/c current is seen. In either way, the IPG is measuring a current going through S1, S2.

The IPG has a circuit inside, that is, with a battery, and has a voltage regulator and current monitoring impedance across it. It has also an op-amp that is driving to a constant, e.g., 5 v reference or any voltage, 2 volts, 3 volts etc. It is assumed it is 5 volts in the preceding example. The op-amp drives the output impedance of S2 to a constant current for a short period, while this measurement is being performed. The measurement takes a small fraction of a millisecond to perform, e.g., 100 microseconds.

So, one skilled in the art will understand that a voltage regulator that supplies a steady voltage on S1 and S2, even though current is varying according to the a/c signal driven on the chips and a current measuring circuit in the loop, i.e., measuring a current going (going to S1 and S2), and this is the current that is the measurement of the impedance between chip 0 and chip 1. In one aspect, two chips are on the same bus, e.g., S1 and S2.

If switching it in a square fashion, the current in between S1,S2 is roughly constant because the current in either case is going from S2 to S1, goes via Chip 1 then Chip 0 or is just going through chip 0 and then to chip 1. Either way, the current stays constant, if two different buses are involved.

One skilled in the art will understand that a voltage regulator provided in the can supplies a steady voltage on S1, S2 even though the current is varying according to the AC signal driven on the chips and a current measuring circuit in that loop that is measuring the current that is going through S1, S2 and it is that current that is the measurement of the signal, of the impedance, between chip 0 and chip 1.

If there are two different buses involved, then on one bus of two wires, the current is going from S2 through chip 1 and S1 in one phase, but in the opposite phase the current is returning from, in one phase, the current is going from S2 through chip 1 and out the electrodes and not returning through S1 and in the opposite phase, the current is going from the electrodes through the op-amp back to S1 and as the current is returning in S1 but not S2.

An aspect includes a modification of a four point probe technology. For example, a four point circuit includes separated, but local, closed loops in which the power supply to the op-amp is being supplied by both.

1. An apparatus comprising at least one implantable lead, the apparatus comprising:
at least two satellites electrically coupled to a common implantable pulse generator by at least two conductors extending from the common implantable pulse generator to each of the at least two satellites, each satellite is associated with a respective tissue site, the at least two satellites comprising:
a first satellite associated with a first tissue site; and
a second satellite associated with a second tissue site, each satellite comprising:
an integrated circuit coupled to the two conductors and having a differential amplifier;
a first electrode communicatively coupled to an output of the integrated circuit, the first electrode selectively associated with a first portion of a tissue site; and
a second electrode adjacent to the first electrode communicatively coupled to an input of the integrated circuit, the second electrode selectively associated with a second portion of a tissue site,
wherein the respective differential amplifier generates predetermined voltages between the second electrode of the first satellite and the second electrode of the second satellite enabling measurement of electrical impedance of tissue substantially between the first tissue site and the second tissue site.

2. The apparatus of claim 1, wherein the voltage on the second electrode of a particular satellite provides feedback to the differential amplifier which produces a drive voltage on the first electrode of the particular satellite.

3. The apparatus of claim 1, wherein the second input of the differential amplifier in a particular satellite is a reference parameter generated by the corresponding integrated circuit.

4. The apparatus of claim 3, wherein the parameter comprises at least one of a reference voltage or a reference current.

5. (canceled)

6. The apparatus of claim 3, wherein the reference parameter in the first satellite is a parameter substantially equivalent to and of opposite polarity to a reference parameter of the second satellite.

7. The apparatus of claim 3, wherein the reference parameter in the first satellite is a time-varying reference parameter and the reference parameter of the second satellite is a substantially non-time-varying reference voltage.

8. The apparatus of claim 3, wherein the time-varying reference parameter comprises at least one of a sine wave, a triangle wave, or a square wave.

9-10. (canceled)

11. The apparatus of claim 7, wherein the reference parameter comprises at least one of a voltage that is a multiple of a diode drop or a voltage of one diode drop.

12. (canceled)

13. The apparatus of claim 1, wherein the at least one electrode comprises a segmented electrode.

14. (canceled)

15. A system comprising:
an implantable pulse generator;
at least one implantable lead comprising:
at least two satellites electrically coupled to the implantable pulse generator by at least two conductors extending from the implantable pulse generator to each of the at least two satellites, each satellite associated with a respective tissue site, the at least two satellites comprising:
a first satellite associated with a first tissue site; and
a second satellite associated with a second tissue site, each satellite comprising:
an integrated circuit coupled to the two conductors and having a differential amplifier;
a first electrode communicatively coupled to an output of the integrated circuit, the first electrode selectively associated with a first portion of a tissue site; and
a second electrode adjacent to the first electrode communicatively coupled to an input of the integrated circuit, the second electrode selectively associated with a second portion of a tissue site,
wherein the respective differential amplifiers generate predetermined voltages between the second electrode of the first satellite and the second electrode of the second satellite enabling measurement of electrical impedance of tissue substantially between the first tissue site and the second tissue site.

16. The system of claim 15, wherein the second input of the differential amplifier in a particular satellite is a reference parameter generated by the corresponding integrated circuit.

17. The system of claim 16, wherein the reference parameter comprises at least one of a reference voltage or a reference current.

18. (canceled)

19. The system of claim 15, wherein the common implantable pulse generator comprises a current sense circuit that measures the time-varying current between the two conductors.

20. The system of claim 15 wherein the common implantable pulse generator comprises a voltage sense circuit that measures the time-varying voltage between the two conductors.

21. The system of claim 15, wherein the pulse generator comprises:
an analog-to-digital convertor communicably associated with the integrated circuit;
and an amplifier communicably associated with the analog-to-digital convertor.

22. An apparatus comprising:
at least one implantable lead comprising:
at least two satellites electrically coupled to a power source by at least two conductors extending from the power source to each of the at least two satellites, each satellite associated with a respective tissue site, the at least two satellites comprising:
a first satellite associated with a first tissue site; and
a second satellite associated with a second tissue site, each satellite comprising:
an integrated circuit coupled to the two conductors and having a variable current regulator and a voltage sampling circuit;
a first electrode communicatively coupled to an output of the variable current regulator, the first electrode selectively associated with a first portion of a tissue site;
a second electrode adjacent to the first electrode communicatively coupled to an input of the voltage sampling circuit, the second electrode selectively associated with a second portion of a tissue site,
wherein the voltage sampling circuits are capable of transmitting data representing the voltages, the voltages from the first satellite and the second satellite represent the electrical impedance of tissue substantially between the first tissue site and the second tissue site.

23. The apparatus of claim 22, wherein the variable current in the first satellite is substantially equivalent but of opposite polarity to a variable current of the second satellite.

24. The apparatus of claim 22, wherein the power source provides a signal that synchronizes the current sources on each of the two satellites.

25. The apparatus of claim 22, wherein the variable current in the first satellite is regulated and the variable current in the second satellite is not regulated.

26. The apparatus of claim 25, wherein the first current source varies independently of the second satellite.

27. A method for use with an apparatus comprising at least two satellites electrically coupled to a common implantable pulse generator by at least two conductors extending from the common implantable pulse generator to each of the at least two satellites, each satellite is associated with a respective tissue site, the at least two satellites comprising a first satellite associated with a first tissue site, and a second satellite associated with a second tissue site, each satellite comprising an integrated circuit coupled to the two conductors and having a differential amplifier; and a first electrode communicatively coupled to an output of the integrated circuit, the first electrode selectively associated with a first portion of a tissue site; and a second electrode adjacent to the first electrode communicatively coupled to an input of the integrated circuit, the second electrode selectively associated with a second portion of a tissue site, the method comprising the steps of:
generating predetermined voltages between the second electrode of the first satellite and the second electrode of the second satellite enabling measurement of electrical impedance of tissue substantially between the first tissue site and the second tissue site.

28. A system comprising:
an implantable pulse generator, the implantable pulse generator comprising:
a variable current source capable of providing a time-varying current through each of two conductors;
at least one implantable lead comprising:
at least two satellites electrically coupled to the pulse generator by at least two conductors extending from the implantable pulse generator to each of the at least two satellites, each satellite associated with a respective tissue site, wherein:
a first satellite of the at least two satellites associated with a first tissue site, the first satellite comprising:
an integrated circuit coupled to the two conductors and having a voltage sampling circuit;
a first electrode communicatively connected to a first conductor, the first electrode selectively associated with a first portion of the first tissue site; and
a second electrode adjacent to the first electrode communicatively coupled to an input of the voltage sampling circuit, the second electrode selectively associated with a second portion of the first tissue site
a second satellite of the at least two satellites associated with a second tissue site, the second satellite comprising:
an integrated circuit coupled to the two conductors and having a voltage sampling circuit;
a third electrode communicatively connected to a second conductor, the third electrode selectively associated with a first portion of the second tissue site; and
a fourth electrode adjacent to the third electrode and communicatively coupled to an input of the voltage sampling circuit of the second satellite, the fourth electrode selectively associated with a second portion of the second tissue site,
wherein the voltage sampling circuits are capable of transmitting data representing the voltages, the voltages from the first satellite and the second satellite representing the electrical impedance of tissue substantially between the first tissue site and the second tissue site.

29-31. (canceled)
